(19) United States
(12) Patent Application Publication
(10) Pub. No.: US 2015/0276608 A1
(43) Pub. Date: Oct. 1, 2015

(54) BIOMOLECULE DETECTION METHOD AND BIOMOLECULE DETECTION APPARATUS

(71) Applicant: Sony Corporation, Tokyo (JP)

(72) Inventors: Yusaku Nakashima, Tokyo (JP); Takuya Kishimoto, Tokyo (JP); Akio Yasuda, Tokyo (JP); Noriyuki Kishii, Kanagawa (JP); Kazuki Ikeshita, Tokyo (JP)

(73) Assignee: Sony Corporation, Tokyo (JP)

(21) Appl. No.: 14/665,465

(22) Filed: Mar. 23, 2015

(30) Foreign Application Priority Data
Mar. 31, 2014 (JP) 2014-074608

(51) Int. Cl. G01N 21/65; G01N 33/574

(52) U.S. Cl. CPC G01N 21/65; G01N 33/57492; G01N 2201/06113

(57) ABSTRACT

According to an embodiment of the present disclosure, there is provided a biomolecule detection apparatus including a light emission unit, a measuring unit and an analysis unit. The light emission unit is configured to emit excitation light to living cells, the living cells having been in contact with an antitumor drug in advance. The measuring unit is configured to measure a Raman spectrum of the living cells. The analysis unit is configured to analyze whether or not the antitumor drug and a target biomolecule are bound with each other on the surface or inside of the living cells, based on the Raman spectrum.

### CROSS REFERENCE TO RELATED APPLICATIONS

This application claims the benefit of Japanese Priority Patent Application JP 2014-074608 filed Mar. 31, 2014, the entire contents of which are incorporated herein by reference.

### BACKGROUND

The present disclosure relates to biomolecule detection methods and biomolecule detection apparatuses, and more specifically, to techniques of analyzing in living cells whether or not an antitumor drug and a target biomolecule are bound with each other, based on a Raman spectrum, and the like.

It has become clear that certain biomolecules may be highly expressed in tumor cells as compared to those in normal cells. Therapeutic drugs targeting such biomolecules have been developed. Besides, so far, methods of detecting those biomolecules having been expressed in tissues and cells have also been developed.

For example, Japanese Patent Application Laid-open No. 2008-298654 (hereinafter referred to as Patent Document 1) discloses a “method for detecting a plurality of target molecules in a test sample” regarding samples obtained from patients suffering from some diseases including cancer. According to this method, a target molecule such as proteins is detected by being labeled with a metal label or other coloring labels.

Further, for example, Japanese Patent Application Laid-open No. 2008-295328 (hereinafter referred to as Patent Document 2) discloses a cancer detection method which detects a gene alteration in a cancer tissue by using a DNA chip method, a Southern blot method, a Northern blot method, a real-time RT-PCR method, a FISH method, a CGH method, an array CGH method, a bisulfite sequencing method, or a COBRA method.

### SUMMARY

In cases where the labels as described in Patent Document 1 are used, preparation of specimens has been complicated, by such as fixing the tissue in advance and preparing slices, in order to observe the specimens and check the presence or absence of the target molecule. On the other hand, in order to carry out the above-mentioned methods in Patent Document 2, it would need extracting nucleic acids from the cells, and preparing slices.

In view of the above circumstances, it is desirable to provide an apparatus capable of detecting a biomolecule that serves as a drug target of an antitumor drug, in living cell state.

According to an embodiment of the present disclosure, there is provided a biomolecule detection apparatus including a light emission unit, a measuring unit and an analysis unit. The light emission unit is configured to emit excitation light to living cells, the living cells having been in contact with an antitumor drug in advance. The measuring unit is configured to measure a Raman spectrum of the living cells. The analysis unit is configured to analyze whether or not the antitumor drug and a target biomolecule are bound with each other on the surface or inside of the living cells, based on the Raman spectrum.

The analysis unit may compare an intensity of a specific peak of the Raman spectrum with a predetermined reference value.

The Raman spectrum may be obtained by measuring while scanning a position at which the excitation light is emitted. The analysis unit may analyze whether or not the antitumor drug and the target biomolecule are bound with each other based on information of the position at which the excitation light is emitted; each Raman spectrum with respect to a corresponding position, in a plurality of different positions at each of which the excitation light is emitted; and information of a predetermined distribution of the target biomolecule.

The Raman spectrum may be obtained by separating nonlinear Raman scattered light. The excitation light may include pump light. The pump light may have a wavelength of 700 nm or more and 1500 nm or less.

The excitation light may include probe light. The probe light may be set at a wavelength such that a Raman band deriving from the antitumor drug appears within a range of 2000 cm-1 or more and 2300 cm-1 or less.

The target biomolecule may include a protein forming a receptor.

The antitumor drug may have a triple bond. The analysis unit may analyze based on a peak deriving from the antitumor drug in the Raman spectrum.

The antitumor drug may have an axial substituent.

The living cells may be cells having been in contact with the antitumor drug in a state of being surrounded by a clathrate.

The clathrate may have a cyclic structure made by a sugar chain. The clathrate may bind to a receptor expressed in tumor cells.

Further, the living cells may be cells having been in contact with the antitumor drug in a state of being surrounded by a clathrate having a triple bond; and the analysis unit may analyze based on a peak deriving from the clathrate in the Raman spectrum.

The clathrate may have an axial substituent.

According to another embodiment of the present disclosure, there is provided a biomolecule detection method including a light emission process, which is emitting excitation light to living cells, the living cells having been in contact with an antitumor drug in advance; a measuring process, which is measuring a Raman spectrum of the living cells; and an analysis process, which is analyzing whether or not the antitumor drug and a target biomolecule are bound with each other on the surface or inside of the living cells, based on the Raman spectrum.

According to an embodiment of the present disclosure, an apparatus capable of detecting a biomolecule that serves as a drug target of an antitumor drug, in living cell state, can thus be provided. Note that the effects described above are not limitative; and any effect described in the present disclosure may be produced.

These and other objects, features and advantages of the present disclosure will become more apparent in light of the following detailed description of best mode embodiments thereof, as illustrated in the accompanying drawings.

### BRIEF DESCRIPTION OF DRAWINGS

FIG. 1 is a schematic diagram showing an example of a biomolecule detection apparatus of a first embodiment of the present disclosure;

FIG. 2 is a flowchart showing processes of a biomolecule detection method using the biomolecule detection apparatus of the first embodiment;

FIG. 3 is a figure for explaining a difference between spontaneous Raman scattered light and coherent anti-Stokes Raman scattering (CARS);

FIGS. 4A and 4B are schematic diagrams showing an example of a biomolecule detection apparatus of a second embodiment of the present disclosure;

FIG. 5 is a flowchart showing processes of a biomolecule detection method using the biomolecule detection apparatus of the second embodiment;

FIG. 6A is a graph showing a Raman spectrum deriving from albumin;

FIG. 6B is a graph showing a Raman spectrum deriving from erlotinib; and

FIG. 7 is a graph showing Raman spectra of Test Examples 1 to 4 in Experimental Example 2.

### DETAILED DESCRIPTION OF EMBODIMENTS

Hereinafter, favorable embodiments for carrying out the teachings of the present disclosure will be described. Note that the following description of the embodiments illustrates certain representative embodiments of the present disclosure; and it is not to be construed as limiting the scope of the present disclosure.

1. Biomolecule Detection Apparatus of First Embodiment of Present Disclosure

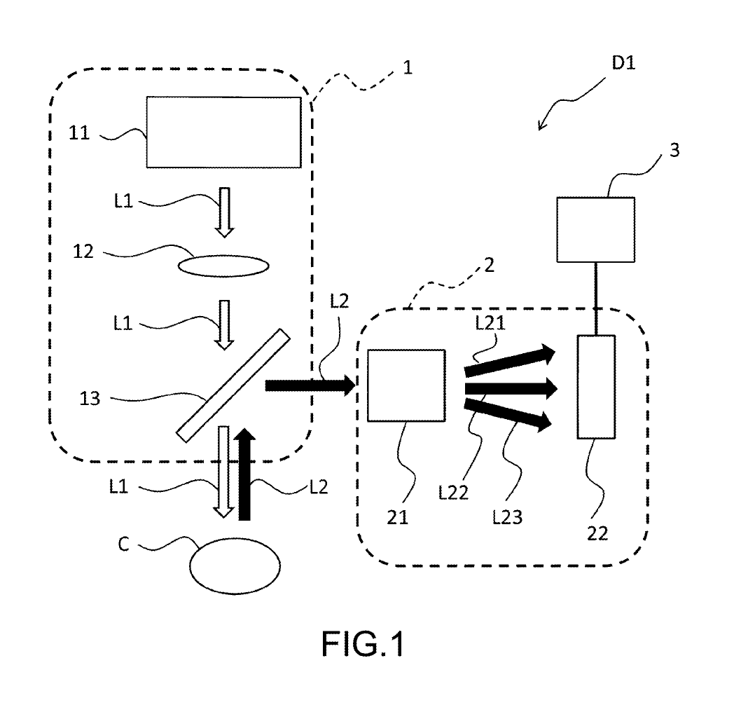

A biomolecule detection apparatus of a first embodiment of the present disclosure will be described. FIG. 1 is a schematic diagram showing an example of configuration of the biomolecule detection apparatus of the first embodiment. The biomolecule detection apparatus denoted by the reference symbol “D1” in FIG. 1 has a light emission unit 1 configured to emit excitation light to living cells C; a measuring unit 2 configured to measure a Raman spectrum of the living cells C; and an analysis unit 3 configured to analyze whether or not an antitumor drug and a target biomolecule are bound with each other on the surface or inside of the living cells C, based on the Raman spectrum. The living cells C are cells that have been in contact with the antitumor drug in advance. With reference to FIG. 1, the components of the biomolecule detection apparatus D1 will be described in order.

(Light Emission Unit)

The light emission unit 1 is a component for emitting the excitation light to the living cells C. The configuration of the light emission unit 1 is not limited as long as it is capable of emitting the excitation light to the living cells C; and known configuration can be employed. For example, the light emission unit 1 includes a light source 11 which emits the excitation light (arrow L1). Any configuration known as an excitation light source may be employed as the light source 11; and for example, a laser light source can be employed. Further, in order to measure nonlinear Raman scattered light in a biomolecule detection method which will be described later; a pulse laser generator may be employed as the light source 11. In addition, by providing an optical fiber to the light emission unit, it may make it possible to guide the excitation light to a lesion in vivo.

The light emission unit 1 may further have an objective lens 12 to collect the excitation light output from the light source 11 and apply the collected excitation light to the living cells C. In addition, for example, a dichroic mirror 13 may be provided between the light source 11 and the living cells C. With the dichroic mirror 13 which allows light to pass therethrough or be reflected depending on wavelengths of the light, it may make it possible to separate the excitation light with reflected light having the same wavelength as the excitation light from Raman scattered light, and to allow the Raman scattered light to enter the measuring unit 2 which will be described later.

The biomolecule detection apparatus D1 may also be configured to be capable of simultaneously emitting the excitation light to a plurality of positions in a specimen containing the living cells C; by having the light emission unit 1 provided with a plurality of light sources 11 and the like. In addition, in cases where such a configuration is employed in the light emission unit 1, it may be desirable to also provide a plurality of spectroscopes 21, a plurality of photodetectors 22 and the like, to the measuring unit 2 which will be described later; in order to make it possible to measure the Raman spectrum based on the Raman scattered light from each of the positions at which the excitation light is emitted.

(Measuring Unit)

The measuring unit 2 is a component for measuring the Raman spectrum of the living cells C, to which the excitation light emitted by the light emission unit 1 is applied. The configuration of the measuring unit 2 is not limited as long as it is capable of measuring the Raman spectrum of the living cells C; and known configuration can be employed. For example, the measuring unit 2 may include a spectroscope 21 and a photodetector 22. The spectroscope 21 may have, for example, a spectroscopic element such as a diffraction grating and a prism. The spectroscope 21 allows the light including the entered Raman scattered light (L2) to be spatially dispersed depending on wavelengths thereof. The photodetector 22 detects the light separated by the spectroscope 21 (arrows L21, L2 and L23 in FIG. 1). As the photodetector 22, for example, a two-dimensional array photodetector such as a two-dimensional charge coupled device (CCD) having pixels arranged in an array may be employed.

(Analysis Unit)

The analysis unit 3 is a component for analyzing whether or not the antitumor drug and the target biomolecule are bound with each other on the surface or inside of the living cells C, based on the Raman spectrum obtained by the measurement by the measuring unit 2. The analysis unit 3 may be made up of, for example, a general-purpose computer including a central processing unit (CPU), a memory, a hard disk, an interface and the like.

The above-described biomolecule detection apparatus D1 may also have, for example, an input unit (not shown in FIG. 1) for allowing a user to input a value such as a reference value which will be described later, a display unit for displaying a result indicating whether or not the target biomolecule has been detected (not shown in FIG. 1), and the like.

2. Detection of Biomolecule by Using Biomolecule Detection Apparatus of First Embodiment of Present Disclosure

A detection of the biomolecule by using the biomolecule detection apparatus D1 of the first embodiment will be described. In other words, an example of a biomolecule detection method according to the present disclosure will be described. First, the living cells C that serves as a specimen will be described.

The living cells C herein are cells that are in the state of performing vital actions such as respiration. More specifically, the cells fixed by alcohols or formalin are not the “living cells”. The cells fractured by supersonic waves, a homogenizer or the like are not the “living cells” either. The living cells may include, for example, cells, tissues or an organ, collected from a living body in advance. In this case, a living state of the cells may be maintained for a certain time by preserving the cells with the use of physiological saline or a buffer solution. Furthermore, the living cells C may be cultured from those cells or the cells obtained from those tissues or the organ. Moreover, the living cells C may be those present in the in vivo state, if it is possible to apply the excitation light thereto and measure the Raman scattered light therefrom, as will be described later. Note that the biomolecule detection method according to the present disclosure may include performing processes of the biomolecule detection method in a living cell state after the living cells have been in contact with the antitumor drug which will be described later; and it does not mean to exclude the biomolecule detection method including a process of fixing the living cells C by alcohols or formalin before performing those processes.

Further, the living cells C herein may include tumor cells. The living cells C may also include cells suspected of being tumor cells. In addition, an abundance ratio of normal cells, tumor cells, and cells suspected of being tumor cells is not limited in particular but may be any ratio. The “tumor cells” are, for example, the cells deriving from a lesion determined to be containing a tumor, found based on clinical findings, a known disease marker, or the like, in a human or an animal. Moreover, the already established cell lines derived from the tumor cells may also be regarded as the tumor cells, in the biomolecule detection method according to the present disclosure.

The living cells may be in a state where the cells are bound to each other, or a state where the cells are detached from each other. Examples of the state where the cells are bound to each other include organs, tissues and the cells collected from them. Examples of the state where the cells are detached from each other include blood cells and the like.

The tumor cells may include cells derived from any types of tumors, including epithelial tumors such as squamous epithelium and glandular epithelial; and non-epithelial tumors such as connective tissue, blood vessel, hematopoietic tissue, muscle tissue and neural tissue. The epithelial tumors include carcinoma; the non-epithelial tumors include sarcoma; and examples of tumors of hematopoietic tissues include leukemia. The tumor cells may include mixed tumor which is a combination of any of the above. Examples of carcinoma include gastrointestinal cancer, prostate cancer, ovarian cancer, breast cancer, head and neck cancer, lung cancer, non-small-cell lung cancer, cancer of nervous system, kidney cancer, retina cancer, skin cancer, liver cancer, pancreatic cancer, genital-urinary cancer, bladder cancer, and the like. Further, the tumor cells may be those of primary tumor, or for example, metastatic tumor such as cancer with metastasis to peritoneum and lymph nodes.

The above-mentioned living cells C are the cells that have been in contact with the antitumor drug in advance. In other words, the living cells C are those having been in contact with the antitumor drug, before the excitation light is emitted to the living cells C in a light emission process which will be described later. The antitumor drug is a medical agent that binds to a specific biomolecule as a target on the surface or inside of cells, after being in contact with the living cells. Herein, the specific biomolecule to which the antitumor drug binds will be referred to as the “target biomolecule”. Accordingly, when the target biomolecule exists in the living cells C, it would be in the state where the antitumor drug is bound to the target biomolecule, at the stage of emission of the excitation light thereto. Furthermore, in cases where the target biomolecule exists abundantly in the tumor cells, the amount of target to be bound to the antitumor drug at the surface or inside of the cells would be increased, so the antitumor drug would be in a concentrated state in the surface or inside of the cells. As a result, for example, in cases where the antitumor drug is added to a culture fluid, the concentration of the antitumor drug becomes higher in the surface or inside of the living cells than the concentration in the culture fluid. In cases where the antitumor drug is added to blood in vivo, the concentration becomes higher in the surface or inside of the living cells than the concentration in the blood.

The term “target biomolecule” as used herein includes molecules in general which may be synthesized, metabolized or accumulated in vivo, as long as the molecule specifically binds to any antitumor drug. Examples of such biomolecules include nucleic acids such as DNA and RNA; peptides; proteins; lipid-protein complexes; and the like. Further, the biomolecule as the target of the antitumor drug may be any biomolecule, depending on the antitumor drug that is selected. In addition, for example, the biomolecule may exist on the surface of a cell membrane; may exist inside the cell; or may exist in both the inside and outside the cell by penetrating the cell membrane, like a receptor.

In the biomolecule detection method according to the present disclosure, the antitumor drug binds to the biomolecule as the target, on the surface or inside of the cells. Since the specific biomolecule which serves as the target biomolecule of the antitumor drug is highly expressed in the tumor cells, the antitumor drug would be concentrated in the surface or inside of the tumor cells as compared to the concentration thereof in the culture fluid or in the blood. It thus makes it possible to measure the Raman scattered light deriving from the antitumor drug at higher intensity, in a measuring process which will be described later.

In addition, the antitumor drug may be, for example, a signaling inhibitor which inhibits activation of a signaling pathway. The signaling inhibitor binds to the target biomolecule, to inhibit signaling that is mediated by the target biomolecule. Accordingly, the biomolecule that serves as the target of the signaling inhibitor may be a protein involved in signaling, or the like. Further, among signal inhibitors, the antitumor drug may be a kinase inhibitor. Kinase inhibitors include tyrosine kinase inhibitors and serine-threonine kinase inhibitors. Examples of tyrosine kinase inhibitors include receptor tyrosine kinase inhibitors and non-receptor tyrosine kinase inhibitors. Examples of serine-threonine kinase inhibitors include mTOR inhibitors. Besides, the antitumor drug may be a tubulin inhibitor.

In the biomolecule detection method according to the present disclosure, for example, an antitumor drug targeting a receptor-type kinase may be employed. Examples of the receptor of the receptor-type kinase include an epidermal growth factor receptor (EGFR), a human epidermal growth factor receptor 2 (Her2), an insulin-like growth factor 1 receptor (IGF1R), a vascular endothelial growth factor receptor (VEGFR), a platelet-derived growth factor receptor (PDGFR), a fibroblast growth factor receptor (FGFR), a colony stimulating factor 1 receptor (CSF1R), a stem cell factor receptor (c-Kit), a hepatocyte growth factor receptor (c-Met), a human Fms-like tyrosine kinase 3 receptor (FLT3), a nerve growth factor (NGF) receptor tyrosine kinase (Trk), a Tie2 receptor (Tie2), an activin receptor-like kinase (Alk), a GDNF receptor tyrosine kinase (Ret), and the like. Further, in the biomolecule detection method according to the present disclosure, the target biomolecule of the antitumor drug may be at least one selected from these receptors.

As the above-described antitumor drug, one having a triple bond may be desirable. Since a triple bond is a structure almost not contained in an organism, it makes it easier to distinguish the Raman scattered light deriving from the tumor cells from the Raman scattered light deriving from the biomolecule, in the detection of the biomolecule, which will be described later. Examples of the triple bond include functional groups such as an axial substituent, a nitrile group and an isonitrile group. Further, one having an axial substituent may be desirable as the antitumor drug.

The above-described antitumor drug may be, for example, a molecularly-targeted treatment drug. A molecularly-targeted drug is a medical agent which inhibits a function of a target, the target being a biomolecule that is known to be highly expressed in tumor cells, which biomolecule may be a product of a cancer gene or the like.

Examples of the molecularly-targeted treatment drugs include antibody drugs and small molecule inhibitors. Among the molecularly-targeted drugs, the drugs which are also called small molecule inhibitors or small molecule compounds are substances of several hundred to several thousand Da, which can be easily taken into cells and which can bind to the target biomolecule in the cells. Examples of the small molecule inhibitors include imatinib (Glivec (registered trademark)), gefitinib (Iressa (registered trademark)), erlotinib (Tarceva (registered trademark)), sunitinib (Sutent (registered trademark)), sorafenib (Nexavar (registered trademark)), dasatinib (Sprycel (registered trademark)), nilotinib (Tasigna (registered trademark)) and the like.

The contact between the above-mentioned antitumor drug and the living cells C may be made by any method, which is not limited, as long as it is performed in such a manner that the living cells C and the antitumor drug can be in contact with each other. For example, a solution containing the antitumor drug may be directly applied by dropping on the living cells C. The solution containing the antitumor drug may be sprayed or coated on the living cells C. Furthermore, also by administering the antitumor drug to a living body and allowing the antitumor drug to reach the lesion, the living cells C and the antitumor drug can be in contact with each other.

The concentration of the antitumor drug to be in contact with the living cells C and the time of contact may be appropriately set depending on the nature of the selected antitumor drug and the state of the living cells C or living body. For example, in cases where the erlotinib is to be dropped on the living cells C, it may be desirable to prepare it so that the concentration of the erlotinib becomes 0.1 to 10 μm in a buffer solution in which the living cells C are preserved.

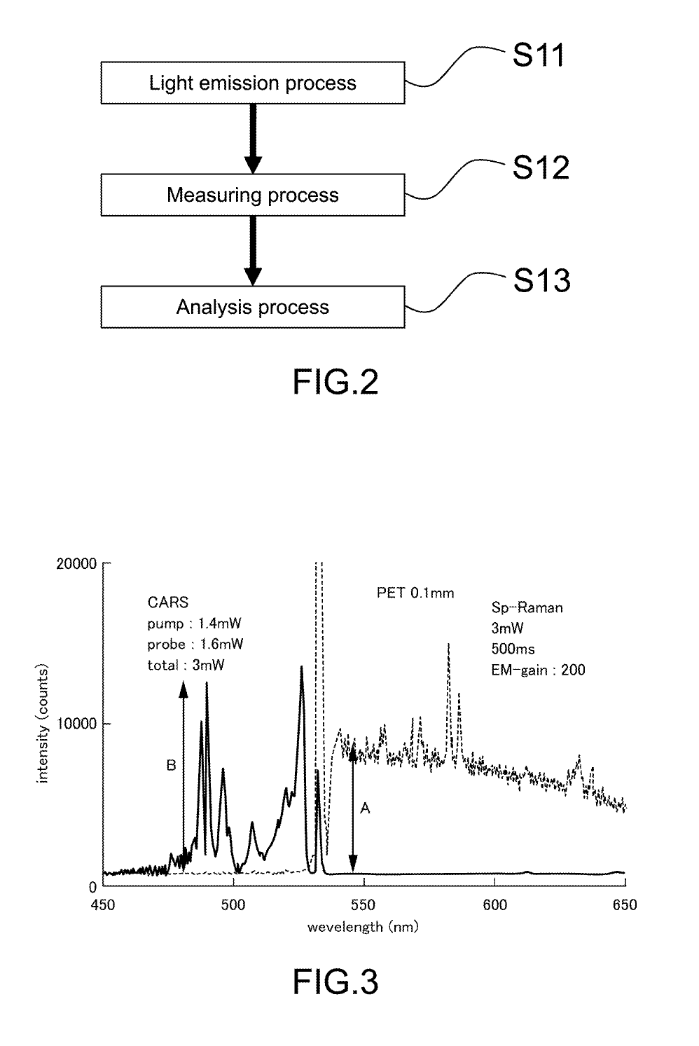

FIG. 2 is a flowchart showing processes of the biomolecule detection method according to the present disclosure. As shown in FIG. 2, the biomolecule detection method includes a light emission process S11, a measuring process S12 and an analysis process S13.

(Light Emission Process)

The symbol S11 in FIG. 2 denotes a light emission process in which the excitation light is emitted by the light emission unit 1 to the living cells C, the living cells C having been in contact with the antitumor drug in advance. In this process S11, the excitation light of a given wavelength is emitted to the living cells C, and the Raman scattered light is produced in the living cells C. In addition, as described above, in cases where the target biomolecule exists in the living cells C, the target biomolecule is in a state where it is bound to the antitumor drug. Therefore, in cases where the target biomolecule exists in the living cells C, the excitation light would be emitted to the antitumor drug as well. The wavelength and output of the emitted excitation light may be any, and may be appropriately set depending on the structure and the nature of the above-described antitumor drug, performance of the light source 11, and the like.

In order to obtain the Raman spectrum of the living cells C to which the excitation light has been applied, in cases where nonlinear Raman spectroscopy which will be described later is used, the excitation light in the process S11 may include pump light. In addition, the excitation light may include probe light. A desirable wavelength of the pump light is 700 nm or more and 1500 nm or less. The light having the wavelength of 700 nm or more and 1500 nm or less has high transmissivity in a living body, so it may enable the excitation light to reach a target located at a deep position in the living body more easily.

Besides, in cases where a Raman band deriving from the antitumor drug is within a range of 2000 cm⁻¹ or more and 2300 cm⁻¹ or less, it would be hardly mistaken for a Raman band deriving from the biomolecule; so this may make it possible to detect the Raman band deriving from the antitumor drug with high precision. It is therefore desirable to set the wavelength of the probe light in such a manner that the Raman band deriving from the antitumor drug appears within a range of 2000 cm⁻¹ or more and 2300 cm⁻¹ or less.

(Measuring Process)

The symbol S12 in FIG. 2 denotes a measuring process in which the Raman spectrum of the living cells C is measured by the measuring unit 2. In this process S12, the Raman spectrum of the living cells C to which the excitation light has been applied by the light emission process S11 is measured. For example, the Raman scattered light being produced from the living cells C can be measured as the Raman spectrum based on the Raman scattered light, by separating the Raman scattered light and detecting it. As described above, in cases where the living cells C are in the state where the antitumor drug is bound with the target biomolecule therein, the Raman scattered light from the antitumor drug would also be measured by the measuring unit 2.

Desirably, the Raman spectrum obtained from the Raman scattered light may be obtained from the nonlinear Raman scattered light. In the nonlinear Raman scattered light, an intensity of the Raman scattered light is nonlinearly enhanced compared to the intensity of the excitation light. Accordingly, this may make it possible to measure the Raman scattered light with better contrast, while an intensity of the Raman scattered light is changed depending on the level of the antitumor drug binding to the target biomolecule in the cells. Examples of the Raman spectrum that can be obtained by separating the nonlinear Raman scattered light include one obtained by induced Raman scattering spectroscopy. Since the induced Raman scattering spectroscopy is a method using amplification of Stokes beams, an intensity of the measured scattered light becomes higher. This makes it possible to detect a molecular vibration deriving from the antitumor drug with higher sensitivity.

Furthermore, the Raman spectrum may be obtained by coherent anti-Stokes Raman scattering (CARS) spectroscopy. In the CARS spectroscopy, anti-Stokes beams would be detected; so the object of the detection would have a shorter wavelength than that of the pump light and the probe light. In cases where a living specimen is used, auto-fluorescence may be produced by the pump light and the probe light (see the arrow A in FIG. 3). Since the auto-fluorescence has about 100 times higher intensity than that of the Raman scattered light, the auto-fluorescence might be a noise that disturbs the detection of the Raman scattered light. With the CARS spectroscopy that measures the anti-Stokes beams of the shorter wavelength side compared to the excitation light, it becomes possible to avoid the noise due to the auto-fluorescence (see the arrow B in FIG. 3). It thus makes it possible to measure the Raman scattered light deriving from the antitumor drug with higher sensitivity.

(Analysis Process)

The symbol S13 in FIG. 2 denotes an analysis process in which the analysis unit 3 analyzes whether or not the antitumor drug and the target biomolecule are bound with each other on the surface or inside of the living cells C, based on the Raman spectrum. In this process S13, for example, the analysis may be made by comparing an intensity of a specific peak of the Raman spectrum measured by the measuring unit 2 with a predetermined reference value. In the following, a case of selecting a peak deriving from the antitumor drug in the Raman spectrum, as the specific peak, and analyzing this peak, will be described as an example.

In this process S13, an intensity of the specific peak deriving from the antitumor drug is compared with the reference value. Selection of the peak deriving from the antitumor drug may be made based on, for example, a wavenumber of a signature peak deriving from the antitumor drug in the Raman spectrum, after determining a Raman spectrum of a specimen containing the antitumor drug alone.

The reference value may be determined by, for example, using cells that are not expressing the target biomolecule as a control, measuring the Raman spectrum after allowing this control to be in contact with the antitumor drug, to determine it from an intensity of light of a wavenumber at which the peak of interest appears. This reference value may also be obtained with respect to the control, every time the Raman spectrum is measured with respect to the living cells C. Moreover, a value which has been previously obtained by measurement with respect to the control may also be used as the reference value.

Besides, in cases where a specimen has a part previously known to have no target biomolecules, this part may be measured to obtain the Raman spectrum; and an intensity of light at a wavenumber range of the signature peak deriving from the antitumor drug in the obtained Raman spectrum may also serve as the reference. Furthermore, it is also possible to measure the Raman spectrum of the living cell C before contacting with the antitumor drug, and use this Raman spectrum for setting the reference value.

The intensity of the peak deriving from the antitumor drug changes depending on the level of the antitumor drug existing on the surface or inside of tumor cells. Accordingly, if the living cells C include the tumor cells, and if the biomolecule is highly expressed in the tumor cells, the intensity of the peak deriving from the antitumor drug in the Raman spectrum would increase due to the antitumor drug being localized on the surface or inside of the cells. As a result, it becomes possible to detect the antitumor drug by the Raman spectrum. Further, with the antitumor drug binding to the target biomolecule, serving as a label, it becomes possible to detect the presence of the biomolecule in the living cells C. The analysis unit 3 may determine that the antitumor drug and the target biomolecule are bound with each other in the surface or inside of the tumor cells if, for example, the intensity of the peak deriving from the antitumor drug is found to be greater than the reference value, as a result of comparison of the intensity and the reference value.

As described above, the biomolecule detection method according to the present disclosure may make it possible to detect the biomolecule as the target of the antitumor drug in the living cells, by obtaining the Raman spectrum of the living cells that have been in contact with the antitumor drug in advance. In this method, since it may not need a process of fixing the cells or a process of extracting the biomolecule, it makes it possible to easily detect the biomolecule that serves as the target.

Furthermore, since the Raman band deriving from the antitumor drug is used in the biomolecule detection method according to the present disclosure, it may make it possible to detect the target biomolecule without using any additional agent or the like for detection of the target biomolecule. The biomolecule that binds to the antitumor drug is a biomolecule that becomes a target in the treatment of a tumor with the use of the antitumor drug. Therefore, detection of the target biomolecule may give useful information in judging effectiveness of treatment and evaluating outcome of treatment.

Besides, regarding the biomolecule as the target of the antitumor drug, since the biomolecule itself is an endogenous biomolecule in a cell, there may be some cases where it is difficult to detect the Raman band deriving from the target biomolecule itself distinctively from those deriving from other biomolecules, by Raman spectroscopy. The biomolecule detection method according to the present disclosure makes it easier to detect a Raman band distinctively from Raman bands deriving from other biomolecules; by detecting the Raman band deriving from the antitumor drug binding to the target biomolecule instead of detecting that deriving from the target biomolecule itself. More particularly, in cases where the antitumor drug has a triple bond, it becomes possible to detect the target biomolecule in the living cells with high precision.

In addition, even in cases where the antitumor drug not bound to the target biomolecule is not sufficiently removed after the contact between the living cells and the antitumor drug, if the target biomolecule exists abundantly in the tumor cells, the intensity of the peak of the Raman band deriving from the antitumor drug would be increased, because the antitumor drug becomes localized on the surface or inside of the tumor cells where relatively large amount of the target biomolecule exists. Accordingly, it becomes possible to detect the target biomolecule with high precision.

According to the above-described biomolecule detection method, since a living cell in which the target biomolecule is detected is a cell having the biomolecule as the target of the antitumor drug highly expressed, the analysis unit may also determine this living cell as a tumor cell, based on the comparison with the reference value. In other words, the biomolecule detection apparatus of the first embodiment may also be used as a tumor cell determining apparatus which determines whether or not the cells are tumor cells.

3. Variation Example of Biomolecule Detection Method of Present Disclosure

In the biomolecule detection method according to the present disclosure, the living cells C may be cells having been in contact with the antitumor drug in a state of being surrounded by a clathrate. In other words, the living cells C may be the cells having been in contact with a clathrate including the antitumor drug in its inside. The clathrate herein is a compound having a hollow space in the center of the molecule, which is capable of incorporating a compound or the like. The antitumor drug surrounded by the clathrate has a much higher affinity with the target biomolecule than an affinity with the clathrate. Accordingly, when this antitumor drug is bound to the target biomolecule, the state of being surrounded by the clathrate is cancelled.

The clathrate may be any, as long as it is capable of surrounding the above-described antitumor drug, and is not limited. For example, one having a cyclic structure made by a sugar chain may be desirable as the clathrate. Examples of compounds as the clathrate having the cyclic structure made by the sugar chain include α-cyclodextrin, β-cyclodextrin, γ-cyclodextrin and the like. For example, since α-cyclodextrin is a compound accepted as a food additive, it may be easily administered to a living body, without a requirement of verification of its toxicity and the like.

Alternatively, a known solubilizing agent may be used as the clathrate. The solubilizing agent may be, for example, an agent to be used when dissolving a hydrophobic agent in a water-soluble solvent. Examples of solubilizing agents include hydroxypropyl-β-cyclodextrin, sulfobutylether-β-cyclodextrin and the like.

The antitumor drug in the state of being surrounded by the clathrate may be prepared by, for example, mixing the antitumor drug and the clathrate; before contacting this antitumor drug with the living cells C. A mixing ratio of the antitumor drug and the clathrate may be appropriately set depending on the nature of the selected antitumor drug and the clathrate. For example, a desirable molar concentration ratio of the antitumor drug and the clathrate may be 1:1 to 1:100.

In the living cells C in which the target biomolecule is expressed, when the antitumor drug in the state of being surrounded by the clathrate is brought into contact with the living cells C, the state of the antitumor drug of being surrounded by the clathrate would be cancelled; and the antitumor drug would bind to the target biomolecule on the surface or inside of the cells. As a result, in the Raman spectrum, the wavenumbers vary between a Raman band deriving from the antitumor drug in the state of being surrounded by the clathrate and a Raman band deriving from the antitumor drug bound to the target biomolecule. By using this change in the wavenumber due to the clathrate, it becomes possible to distinguish the antitumor drug bound to the target biomolecule in the living cells C from the antitumor drug not bound to the target biomolecule, by the Raman spectrum. As a result, it becomes possible to analyze whether or not the antitumor drug and the target biomolecule are bound with each other, with higher precision, by the analysis process S13 with the analysis unit 3. Other effects, which are the same as in the case of using the antitumor drug as described above, may be produced as well.

The clathrate may have the property of binding to a receptor expressed in tumor cells. Such a property may be obtained by, for example, allowing the clathrate to contain a molecule that may bind to the receptor expressed in tumor cells. For example, a receptor to folate is known to be highly expressed in tumor cells. Accordingly, the clathrate may obtain the property of binding to the receptor expressed in tumor cells, by containing the folate. In addition, in order to provide the clathrate with the property of binding to the receptor expressed in tumor cells, the clathrate may contain a structure having high affinity with tumor cells; the structure such as a glucityl group, a glycosyl phenylthiocarbamyl group and a glycosyl pyroglutamyl alanyl group.

By having the property of binding to the receptor expressed in tumor cells, the clathrate may easily get close to the tumor cells. Accordingly, it becomes possible to carry the antitumor drug surrounded by the clathrate to the tumor cells with greater efficiency. Note that, desirably, the receptor expressed in the tumor cells and the target biomolecule of the antitumor drug surrounded by the clathrate are not the same receptors.

Furthermore, the clathrate may have a triple bond in its structure. In other words, the living cells C may be the cells having been in contact with the antitumor drug in a state of being surrounded by a clathrate having a triple bond. In this case, the analysis unit 3 may analyze based on a peak deriving from the clathrate, in the Raman spectrum.

As described above, it would be easy to distinguish the antitumor drug surrounded by the clathrate from the antitumor drug bound to the target biomolecule, by the Raman spectrum. In cases where the clathrate instead of the antitumor drug has the triple bond, by using a change in the wavenumber of the Raman band deriving from the triple bond of the clathrate, it becomes possible to distinguish the clathrate in the state of surrounding the antitumor drug from the clathrate not in the state of surrounding the antitumor drug, by the Raman spectrum. This makes it possible to analyze whether or not the antitumor drug has been released from the state of being surrounded by the clathrate and has bound to the target biomolecule existing in the tumor cells.

Similarly to that of the antitumor drug, examples of the triple bond of the clathrate include functional groups such as an axial substituent, a nitrile group and an isonitrile group. Further, one having an axial substituent may be desirable as the clathrate having the triple bond.

4. Biomolecule Detection Apparatus of Second Embodiment of Present Disclosure

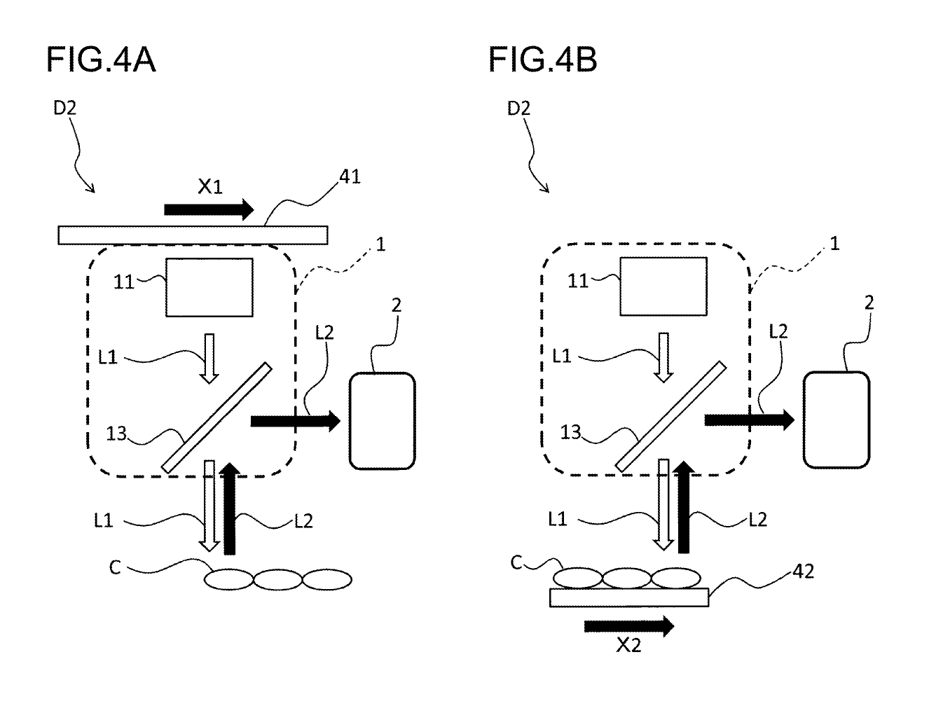

In a biomolecule detection apparatus of a second embodiment of the present disclosure, the Raman spectrum may be obtained by measuring while scanning a position at which the excitation light is emitted. FIGS. 4A and 4B are schematic diagrams showing an example of the biomolecule detection apparatus of the second embodiment of the present disclosure. For example, as shown in FIGS. 4A and 4B, a biomolecule detection apparatus D2 of this embodiment may have a drive mechanism 4 (41 and 42) which changes relative positions of the light emission unit 1 and the living cells C. Note that the illustration of the analysis unit 3 is omitted in FIGS. 4A and 4B. The same configurations as those of the biomolecule detection apparatus D1 of the first embodiment are denoted by the same reference symbols, and they will not be described again.

The drive mechanism 41 shown in FIG. 4A is capable of moving the light emission unit 1 in the direction indicated by an arrow X1 to change the relative positions of the light emission unit 1 and the living cells C, thereby moving the position at which the excitation light is emitted. Further, for example, like the drive mechanism 42 shown in FIG. 4B, the drive mechanism 4 may change the relative positions of the light emission unit 1 and the living cells C by moving the living cells C in the direction indicated by an arrow X2.

Furthermore, the biomolecule detection apparatus D2 of this embodiment may have the drive mechanism 4 (41 and 42) which moves both the light emission unit 1 and the living cells C. In addition, the position at which the excitation light emitted from the light source 11 may be changed by changing an angle of a mirror or the like. The configuration is not limited in particular; as long as it is capable of obtaining the Raman spectrum as one that is measured while scanning the position at which the excitation light is emitted. The configuration that allows scanning the position at which the excitation light is emitted may be, for example, one appropriately employed from known configurations of a scanning microscope or the like.

5. Detection of Biomolecule by Using Biomolecule Detection Apparatus of Second Embodiment

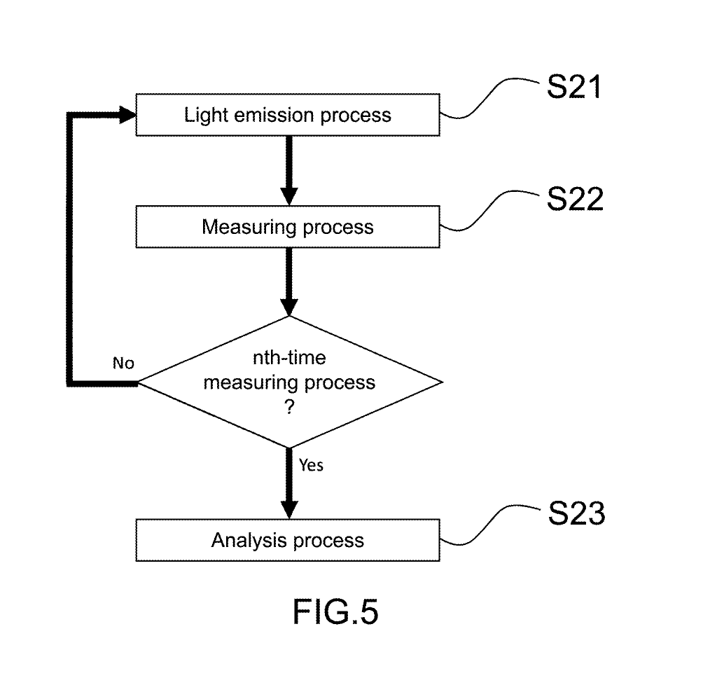

A detection of the biomolecule by using the biomolecule detection apparatus D2 of the second embodiment will be described. In other words, an example of a biomolecule detection method according to the present disclosure will be described. FIG. 5 is a flowchart showing processes of a biomolecule detection method using the biomolecule detection apparatus D2. The biomolecule detection method includes a light emission process S21, a measuring process S22 and an analysis process S23.

The light emission process S21 and the measuring process S22, respectively, are performed in substantially the same manner as the above-described light emission process S11 and the measuring process S12. Further, in the detection of the biomolecule by using the biomolecule detection apparatus D2 of the second embodiment, as shown in FIG. 5, the light emission unit 1 and measuring unit 2 repeats the light emission process S21 and the measuring process S22 while changing the position at which the excitation light is emitted. Thus, the Raman spectrum can be as one that is measured while scanning with the light emission unit or scanning the living cells. Then, for example, after a repetition of the processes for a predetermined number of times (nth time), the measuring process S22 by the biomolecule detection apparatus D2 is ended. Then, in the biomolecule detection apparatus D2, the analysis unit 3 starts the analysis process S23.

The analysis process S23 is able to analyze whether or not the antitumor drug and the target biomolecule are bound with each other, by comparing an intensity of a specific peak in the measured Raman spectra with a predetermined reference value, in substantially the same manner as the above-described biomolecule detection method by using the biomolecule detection apparatus D1 of the first embodiment. The reference value and the specific peak are as described in the above.

In addition, the analysis process S23 is able to analyze whether or not the antitumor drug and the target biomolecule are bound with each other based on information of the position at which the excitation light is emitted; each Raman spectrum with respect to a corresponding position, in a plurality of different positions at each of which the excitation light is emitted; and information of a predetermined distribution of the target biomolecule. The information of the position at which the excitation light is emitted is based on, such as, a distance between each position in the plurality of different positions at each of which the excitation light is emitted.

Besides, the information of the predetermined distribution of the target biomolecule may be, for example, information of localization of the target biomolecule in the cell. Specifically, if the target biomolecule is a membrane protein, the localization is observed on membranes such as cell membranes. The information of the distribution of the target biomolecule may be stored beforehand in memory in the analysis unit 3 or the like, or may be input by the user at the time of starting the analysis process S23.

The analysis unit 3 may be able to plot an intensity of a specific peak to a coordinate based on the above-described information of the position at which the excitation light is emitted, thereby creating 3D data indicating intensities of the respective peaks in a plurality of the positions at which the Raman spectra are measured. Moreover, the analysis unit 3 may analyze whether or not the antitumor drug and the target biomolecule are bound with each other on the surface or inside of the living cells by comparing the 3D expanded distribution of the intensities of the peaks with distribution information of a previously-specified target biomolecule.

For example, if the target biomolecule is a membrane protein, whether or not the distribution of the intensities of the peaks in the 3D data matches with a distribution characteristic of a membrane protein is analyzed. If a degree of coincidence, between distribution information of the target biomolecule and the distribution of the intensity of light, exceeds a threshold value, it may be determined that the antitumor drug and the target biomolecule are bound with each other on the surface or inside of the living cells C. Also in the biomolecule detection apparatus D1 of the first embodiment, if Raman spectra have been sequentially measured in a plurality of positions and positional information of the respective positions has been obtained, the above-mentioned 3D data may be created; and it becomes possible to compare the distribution of the intensities of the peaks and the distribution information of the target biomolecule.

In addition, the above-mentioned distribution information of the previously-specified target biomolecule may also be used as auxiliary information for identifying the distribution of the target biomolecule based on the distribution of the intensities of the peaks.

The biomolecule detection apparatus of the second embodiment makes it possible to sequentially measure the Raman spectra with respect to a plurality of positions in a specimen containing the living cells. Then, by comparing the distribution of the intensity of a specific peak in the Raman spectra with the distribution information of the target biomolecule, it makes it possible to find whether or not the distribution of the specific peak matches with the distribution information. Accordingly, it becomes possible to analyze whether or not the antitumor drug and the target biomolecule are bound with each other, with higher precision. Other effects, which are the same as in the case with the biomolecule detection apparatus of the first embodiment as described above, may be produced as well.

Note that the effects described herein are illustrative and not limitative; and other effects may also be produced.

The present disclosure may employ the following configurations.

(1) A biomolecule detection apparatus, including:

a light emission unit configured to emit excitation light to living cells, the living cells having been in contact with an antitumor drug in advance;

a measuring unit configured to measure a Raman spectrum of the living cells; and

an analysis unit configured to analyze whether or not the antitumor drug and a target biomolecule are bound with each other on the surface or inside of the living cells, based on the Raman spectrum.

(2) The biomolecule detection apparatus according to (1), in which

the analysis unit compares an intensity of a specific peak of the Raman spectrum with a predetermined reference value.

(3) The biomolecule detection apparatus according to (1) or (2), in which

the Raman spectrum is obtained by measuring while scanning a position at which the excitation light is emitted, and

the analysis unit analyzes whether or not the antitumor drug and the target biomolecule are bound with each other based on
information of the position at which the excitation light is emitted,
each Raman spectrum with respect to a corresponding position, in a plurality of different positions at each of which the excitation light is emitted, and
information of a predetermined distribution of the target biomolecule.

(4) The biomolecule detection apparatus according to any one of (1) to (3), in which

the Raman spectrum is obtained by separating nonlinear Raman scattered light.

(5) The biomolecule detection apparatus according to (4), in which

the excitation light includes pump light, the pump light having a wavelength of 700 nm or more and 1500 nm or less.

(6) The biomolecule detection apparatus according to (5), in which

the excitation light includes probe light, the probe light being set at a wavelength such that a Raman band deriving from the antitumor drug appears within a range of 2000 cm⁻¹ or more and 2300 cm⁻¹ or less.

(7) The biomolecule detection apparatus according to any one of (1) to (6), in which

the target biomolecule includes a protein forming a receptor.

(8) The biomolecule detection apparatus according to any one of (1) to (7), in which

antitumor drug has a triple bond, and

the analysis unit analyzes based on a peak deriving from the antitumor drug in the Raman spectrum.

(9) The biomolecule detection apparatus according to (8), in which

the antitumor drug has an axial substituent.

(10) The biomolecule detection apparatus according to any one of (1) to (9), in which

the living cells are cells having been in contact with the antitumor drug in a state of being surrounded by a clathrate.

(11) The biomolecule detection apparatus according to (10), in which

the clathrate has a cyclic structure made by a sugar chain.

(12) The biomolecule detection apparatus according to (10) or (11), in which

the clathrate binds to a receptor expressed in tumor cells.

(13) The biomolecule detection apparatus according to any one of (1) to (7), in which

the living cells are cells having been in contact with the antitumor drug in a state of being surrounded by a clathrate having a triple bond; and

the analysis unit analyzes based on a peak deriving from the clathrate in the Raman spectrum.

(14) The biomolecule detection apparatus according to (13), in which

the clathrate has an axial substituent.

(15) A biomolecule detection method, including:

emitting excitation light to living cells, the living cells having been in contact with an antitumor drug in advance;

measuring a Raman spectrum of the living cells; and

analyzing whether or not the antitumor drug and a target biomolecule are bound with each other on the surface or inside of the living cells, based on the Raman spectrum.

### EXAMPLES

### Experimental Example 1

### 1. Measurement of Raman Scattered Light Deriving from Antitumor Drug

In this experimental example, Raman spectra each deriving from an antitumor drug and from a biomolecule were measured, and it was checked if a Raman band deriving from the antitumor drug was able to be detected.

As the antitumor drug in this experimental example, erlotinib was used. The structure of the erlotinib is described as follows. As described in the following, the erlotinib has a triple bond in its structure. As the erlotinib, Erlotinib Hydrochloride which is a product of Santa Cruz Biotechnology, Inc. was used. A 10 mM aqueous solution thereof was prepared. As the biomolecule, albumin was used. As the albumin, a product of Sigma Aldrich Corporation was used. A 10 mg/ml aqueous solution thereof was prepared.

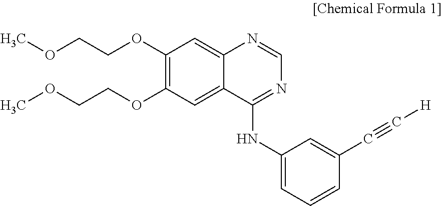 [SMILES: C#Cc1cccc(Nc2ncnc3cc(OCCOC)c(OCCOC)cc23)c1]

The excitation light was made to include pump light of 785 nm, and to include light with the wavenumber of at least a range of 2000 to 2300 cm⁻¹ so that the Raman band of erlotinib of 2300 cm⁻¹ was able to be covered; and thus the Raman spectrum was measured. The excitation light was emitted at the erlotinib and the albumin, Raman scattered light was separated, and the Raman spectra deriving from the respective samples were obtained.

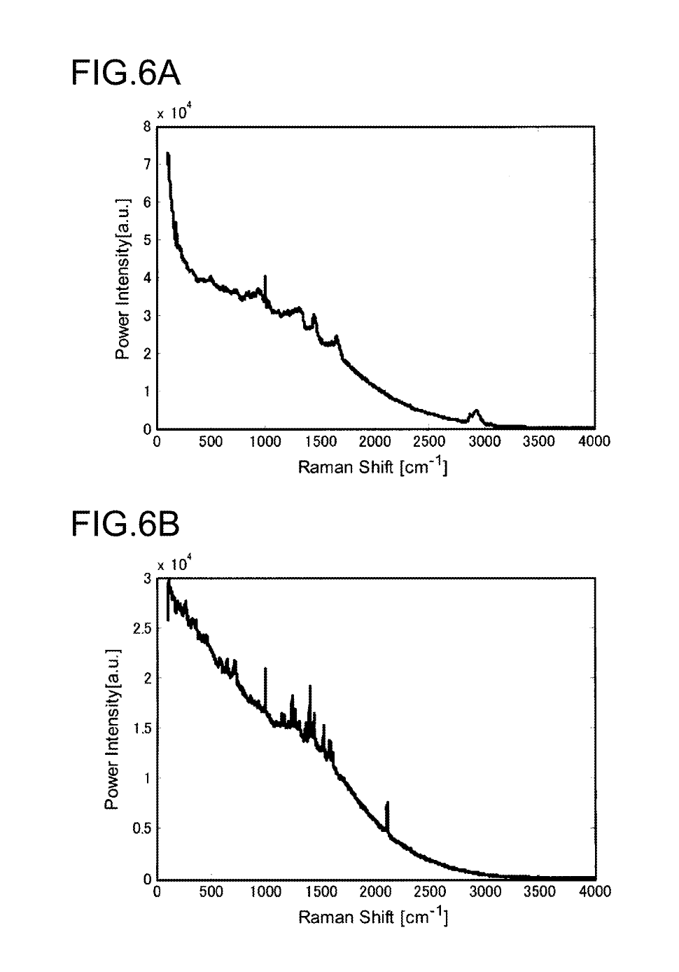

A result of this experimental example is shown in FIGS. 6A and 6B. FIG. 6A shows the Raman spectrum deriving from the albumin. FIG. 6B shows the Raman spectrum deriving from the erlotinib. The abscissa in each of FIGS. 6A and 6B indicates the wavelength of the measured light (Raman shift) and the ordinate indicates the intensity at each wavenumber. As shown in FIG. 6B, a Raman band deriving from a vibration spectrum of the triple bond of the carbons contained in the erlotinib was detected at 2108 cm⁻¹. On the other hand, in the Raman spectrum of the albumin, no Raman band was detected at the wavenumber at which the Raman band of the erlotinib was detected (see FIG. 6A).

From the result of this experimental example, it was confirmed that the Raman shift deriving from the antitumor drug is able to be detected. Specifically, it was revealed that in cases where the structure of the antitumor drug contains the triple bond, the antitumor drug may be detected with higher sensitivity in the Raman spectrum, without overlap of its Raman band with the Raman band deriving from the living body.

### Experimental Example 2

### 2. Detection of Raman Spectrum Light Deriving from Antitumor Drug in Presence of Clathrate

In this experimental example, it was checked if a Raman band deriving from the antitumor drug was to be changed by a clathrate.

As the antitumor drug in this experimental example, erlotinib as in Experimental Example 1 was used. As the clathrate, α-cyclodextrin was used. By using them, Test Examples 1 to 4 were prepared. Test Example 1 was made by dissolving the erlotinib in distilled water at a concentration of 10 μM. Test Example 2 was made by further dissolving the α-cyclodextrin in the Test Example 1, at a concentration of 1 mM. Test Example 3 contained distilled water only. Test Example 4 was made by dissolving the α-cyclodextrin in distilled water at a concentration of 1 mM.

As the excitation light, the pump light and probe light with the same wavelengths as those in Experimental Example 1 were used. The excitation light was emitted at Experimental Examples 1 to 4, Raman scattered light generated from each of the samples was separated, and the Raman spectra were obtained.

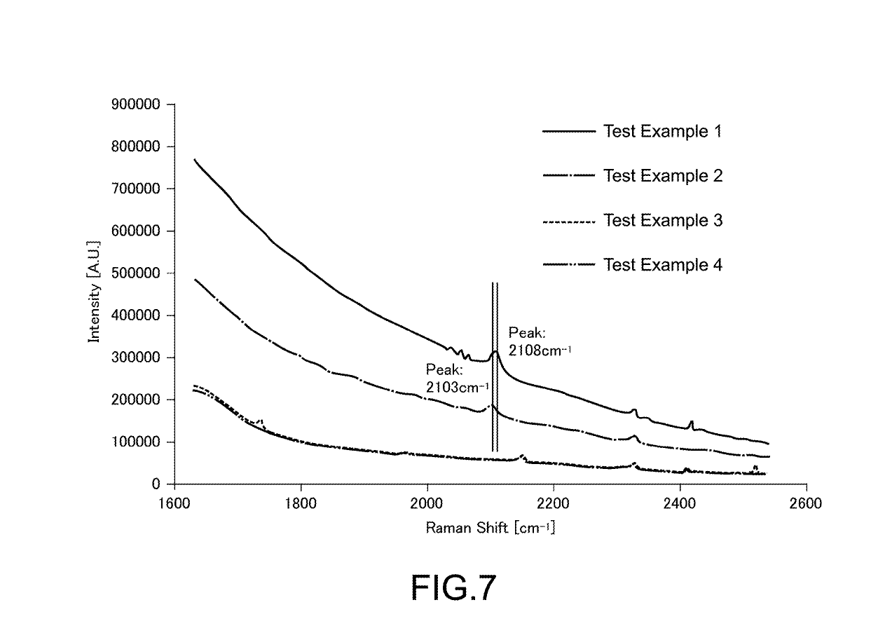

A result of this experimental example is shown in FIG. 7. FIG. 7 shows the Raman spectra obtained from Test Examples 1 to 4. The abscissa in FIG. 7 indicates the wavelength of the measured light (Raman shift) and the ordinate indicates the intensity at each wavenumber. As shown in FIG. 7, regarding Test Examples 1 and 2, the Raman bands deriving from the vibration spectrum of the triple bond of the carbons contained in the erlotinib were detected. In addition, while the Raman band measured for Test Example 1 was 2108 cm⁻¹, the Raman band measured for Test Example 2 was 2103 cm⁻¹. On the other hand, regarding Test Examples 3 and 4 which did not include the erlotinib, no Raman band was detected at the same wavenumber as any of those of Test Examples 1 and 2.

From the result of this experimental example, it was revealed that the Raman band deriving from the antitumor drug differs in wavenumbers between the state of being surrounded by the clathrate and the state of not being surrounded by the clathrate. This result indicates that it becomes possible to distinguish the antitumor drug in the state of being surrounded by the clathrate from the antitumor drug in the state of not being surrounded by the clathrate, by the Raman spectrum, by using the shift of the Raman band.

It should be understood by those skilled in the art that various modifications, combinations, sub-combinations and alterations may occur depending on design requirements and other factors insofar as they are within the scope of the appended claims or the equivalents thereof.

What is claimed is:

1. A biomolecule detection apparatus, comprising:
a light emission unit configured to emit excitation light to living cells, the living cells having been in contact with an antitumor drug in advance;
a measuring unit configured to measure a Raman spectrum of the living cells; and
an analysis unit configured to analyze whether or not the antitumor drug and a target biomolecule are bound with each other on the surface or inside of the living cells, based on the Raman spectrum.

2. The biomolecule detection apparatus according to claim 1, wherein
the analysis unit compares an intensity of a specific peak of the Raman spectrum with a predetermined reference value.

3. The biomolecule detection apparatus according to claim 1, wherein
the Raman spectrum is obtained by measuring while scanning a position at which the excitation light is emitted, and
the analysis unit analyzes whether or not the antitumor drug and the target biomolecule are bound with each other based on
information of the position at which the excitation light is emitted,
each Raman spectrum with respect to a corresponding position, in a plurality of different positions at each of which the excitation light is emitted, and
information of a predetermined distribution of the target biomolecule.

4. The biomolecule detection apparatus according to claim 1, wherein
the Raman spectrum is obtained by separating nonlinear Raman scattered light.

5. The biomolecule detection apparatus according to claim 4, wherein
the excitation light includes pump light, the pump light having a wavelength of 700 nm or more and 1500 nm or less.

6. The biomolecule detection apparatus according to claim 5, wherein
the excitation light includes probe light, the probe light being set at a wavelength such that a Raman band deriving from the antitumor drug appears within a range of 2000 cm⁻¹ or more and 2300 cm⁻¹ or less.

7. The biomolecule detection apparatus according to claim 1, wherein
the target biomolecule includes a protein forming a receptor.

8. The biomolecule detection apparatus according to claim 1, wherein
antitumor drug has a triple bond, and
the analysis unit analyzes based on a peak deriving from the antitumor drug in the Raman spectrum.

9. The biomolecule detection apparatus according to claim 8, wherein
the antitumor drug has an axial substituent.

10. The biomolecule detection apparatus according to claim 1, wherein
the living cells are cells having been in contact with the antitumor drug in a state of being surrounded by a clathrate.

11. The biomolecule detection apparatus according to claim 10, wherein
the clathrate has a cyclic structure made by a sugar chain.

12. The biomolecule detection apparatus according to claim 10, wherein
the clathrate binds to a receptor expressed in tumor cells.

13. The biomolecule detection apparatus according to claim 1, wherein
the living cells are cells having been in contact with the antitumor drug in a state of being surrounded by a clathrate having a triple bond; and
the analysis unit analyzes based on a peak deriving from the clathrate in the Raman spectrum.

14. The biomolecule detection apparatus according to claim 13, wherein
the clathrate has an axial substituent.

15. A biomolecule detection method, comprising:
emitting excitation light to living cells, the living cells having been in contact with an antitumor drug in advance;
measuring a Raman spectrum of the living cells; and
analyzing whether or not the antitumor drug and a target biomolecule are bound with each other on the surface or inside of the living cells, based on the Raman spectrum.
